(19) United States
(12) Patent Application Publication
(10) Pub. No.: US 2013/0001180 A1
(43) Pub. Date: Jan. 3, 2013

(54) STERILE RACK FOR CONTRACEPTIVE PROCEDURE

(76) Inventors: Christopher A. Stout, San Bruno, CA (US); Julian Cruzada, San Jose, CA (US)

(21) Appl. No.: 13/531,305

(22) Filed: Jun. 22, 2012

Related U.S. Application Data
(60) Provisional application No. 61/502,766, filed on Jun. 29, 2011.

(51) Int. Cl. A61B 19/00; F21V 21/00; H05K 7/18

(52) U.S. Cl. 211/85.13

(57) ABSTRACT

A sterile rack is disclosed which includes a plurality of openings, with each opening designed to retain a specific instrument in a substantially vertical position in a medical procedure such a s a transcervical sterilization procedure.

### RELATED APPLICATIONS

This application claims the benefit of the filing date of U.S. Provisional Application Ser. No. 61/502,766, filed on Jun. 29, 2011. The full disclosure of U.S. Provisional Application Ser. No. 61/502,766 is incorporated herein by reference.

### BACKGROUND

Embodiments of the present invention relate to the field of sterile procedures. Specifically embodiments relate to the field of sterile transcervical sterilization procedures.

Conventional sterile procedures are performed using surgical supplies arranged on a flat autoclaveable plastic tray or a flat metal tray often called a “Mayo” tray. A delinted surgical towel is typically placed on the tray before placing instruments and other instruments horizontally on the towel. The towel creates a space between the instruments and the tray, and enhances drying at the end of a sterilization cycle.

Instruments are generally arranged on the tray in the sequence in which they are most commonly used during a specific procedure. This facilitates the exchange of instruments between personnel (e.g. between doctor and assistant) during a procedure. Once an instrument is used it should be returned to the proper location whenever possible. Both the assistants and doctors must generally know all the instruments by name, number, purpose, and sequence of use. During the course of the medical procedure the instruments can become disorganized on the tray, and may become displaced over an edge of the sterile field of the tray.

### SUMMARY

In an embodiment, a sterile rack includes a plurality of openings, with each opening designed to retain a specific instrument in a substantially vertical position in a medical procedure such as a transcervical sterilization procedure. For example, the openings may include a speculum opening, a tenaculum opening, an introducer opening, and a delivery system opening. Other suitable openings include a graspers opening, a hysteroscope opening, and a dilator opening. There may be multiple dilator openings of various diameters. There may also be multiple introducer openings, or multiple delivery system openings.

The sterile rack may be configured to attach to a variety of surfaces. In an embodiment, the sterile rack includes a clamp for clamping to a table top. In an embodiment, the sterile rack includes a clip for clipping onto a rounded table corner. In an embodiment, the sterile rack includes a tubular receptacle to attach to an IV pole. The sterile rack may also include an adjustable arm clamp, and can be made to be collapsible. In an embodiment, the sterile rack is part of an office system also including a laptop, a camera, a light source, and a mobile station such as an IV pole.

### BRIEF DESCRIPTION OF THE DRAWINGS

FIGS. 1A-B illustrate perspective views of a sterile rack in accordance with an embodiment of the present invention.

FIG. 1C illustrates a perspective view of a sterile rack attached to an IV pole in accordance with an embodiment of the present invention.

FIG. 2A illustrates a side-perspective view of the sterile rack including a table clamp in accordance with an embodiment of the present invention.

FIG. 2B illustrates a perspective view of a sterile rack including an adjustable arm claim in accordance with an embodiment of the present invention.

FIG. 3A illustrates a perspective view of a sterile corner rack in accordance with an embodiment of the present invention.

FIG. 3B illustrates a top view of the sterile corner rack of FIG. 3A in accordance with an embodiment of the present invention.

FIG. 4A illustrates a perspective view of a collapsible sterile rack in an expanded configuration in accordance with an embodiment of the present invention.

FIG. 4B illustrates a perspective view of a collapsible sterile rack in a partially collapsed configuration in accordance with an embodiment of the present invention.

FIG. 4C illustrates a perspective view of a collapsible sterile rack in a collapsed configuration in accordance with an embodiment of the present invention.

FIGS. 5A-5B illustrate perspective views of an office system in accordance with an embodiment of the present invention.

### DETAILED DESCRIPTION

Embodiments of the present invention generally provide a sterile rack and office system for carrying out sterile procedures. More specifically, some embodiments provide a sterile rack and office system for a transcervical sterilization procedure.

Various embodiments and aspects of the present invention will be described with reference to details discussed below and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present invention.

In accordance with various embodiments of the present invention, sterile racks are described which may be used to organize and keep sterile instruments substantially vertical. The sterile racks may be formed from a material which can be sterilized, for example in an autoclave, alone or along with the instruments. Such a material may be stainless steel, for example. The sterile racks may also be disposable, and can be formed from recycled materials. Vertical alignment of the sterile instruments may act to prevent the sterile instruments from extending over the edge of the sterile field. The sterile racks may include various custom designed openings for a particular medical procedure. The custom designed openings let the doctor or sterile nurse know immediately if a particular instrument is missing during preparation for the procedure. In addition, the custom designed openings allow the doctor performing the medical procedure to know exactly where the instruments are located. As a result, a sterile nurse may not be needed during the procedure. As the doctor develops her confidence it may be possible for the doctor to know exactly where the instruments are and grab the instruments during the procedure without having to turn around. An area of the sterile rack may also be used for labeling of instruments. For example, the custom designed openings may be labeled corresponding to the particular instrument for which they are designed to receive.

The sterile racks in accordance with embodiments of the present invention may be configured to attach to a variety of different surfaces to assist the doctor during the procedure. For example, the sterile racks can be configured to attach to a table top surface, edge or corner by clamping or clipping means. The sterile racks can also be configured to attach to an IV pole. In an embodiment, the sterile rack is rotatable 360 degrees on the IV pole in order to give access to the instruments.

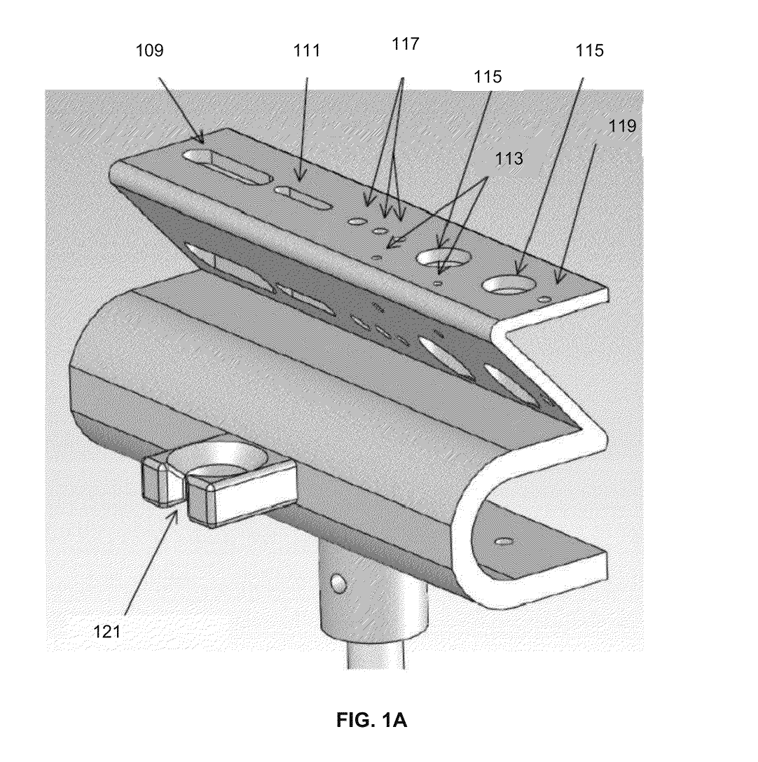

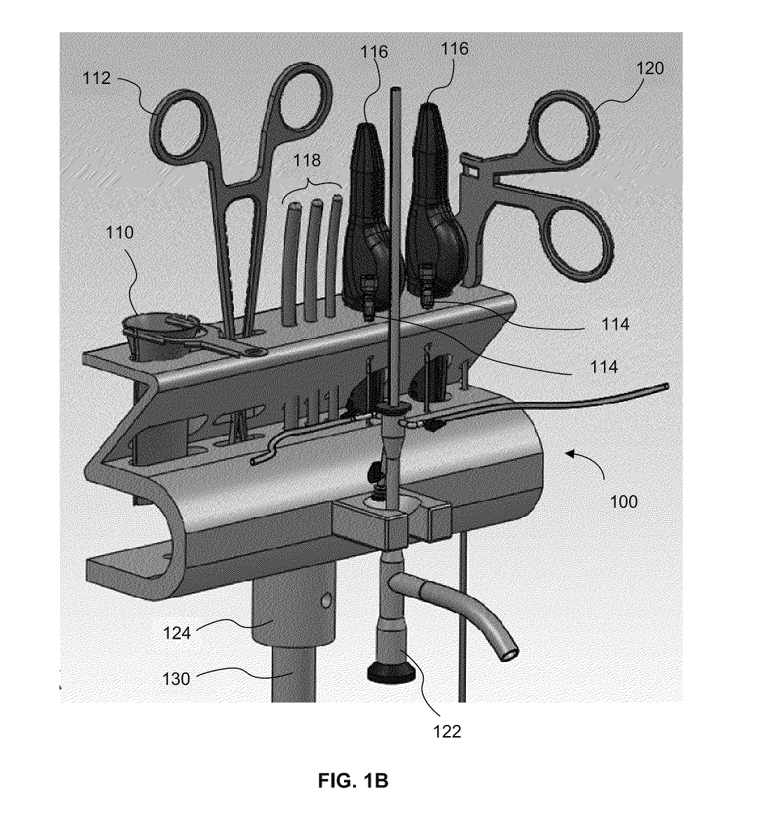

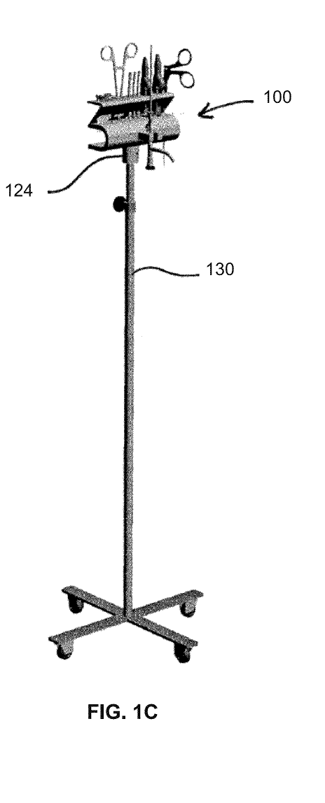

FIGS. 1A-1B illustrate perspective views of a sterile rack in accordance with an embodiment of the present invention. As illustrated, the sterile rack 100 may include a plurality of openings with each opening designed to retain a specific instrument for use in a medical procedure in a substantially vertical position. In an embodiment, the medical procedure is a transcervical sterilization procedure, such as the Essure® procedure (available from Conceptus, Inc. of Mountain View, Calif., United States of America). It is contemplated that the sterile racks in accordance with embodiments of the invention may also be configured for other procedures such as c-sections, OBGYN appointments, child birthing, and other non OBGYN procedures.

In accordance with some embodiments, the sterile rack may include openings vertically aligned in multiple levels of the sterile rack which are separated by an open space. For example, referring to FIGS. 1A-1B, openings 109, 111, 113, 115, and 117 are vertically formed through three levels of the sterile rack 100 to retain and secure instruments 110, 112, 114, 116, and 118, and opening 119 is vertically formed through four levels of the sterile rack 100 to retain instrument 120.

In an embodiment, openings 109, 111, 113 and 115 are provided for principal instruments of the Essure® transcervical sterilization procedure including a speculum 100 for spreading apart tissue, a tenaculum 112 for grabbing and locking onto tissue, an introducer 114 such as the DryFlow™ introducer (available from Conceptus, Inc. of Mountain View, Calif., United States of America) for assisting insertion of a delivery system into a distention valve of a hysteroscope, and a delivery system 116 such as the Essure® delivery system for inserting and delivering a microinsert into each fallopian tube. In an embodiment, there are two openings 115 to accommodate two delivery systems 116 (one for each fallopian tube) and two openings 113 to accommodated two introducers 114. It is contemplated that the sterile rack may additionally include a third delivery system opening 115 and introducer opening 113 to store a back up delivery system and introducer in the event of a failed delivery attempt to a fallopian tube.

Openings 117, 119 and 121 may also be provided in the sterile rack for other instruments that may be utilized during the Essure® procedure such as dilators 118 of various diameters for dilating the cervical opening, graspers 120 which are a long, thin tool which can be slid down the working channel of a hysteroscope to grab tissue, and a hysteroscope 122. A plurality of openings 117 may be included to retain multiple dilators 118 of different diameters such as 2 mm-3 mm, 4 mm-5 mm, and 6 mm-7 mm.

In accordance with some embodiments of the invention, a sterile rack is configured to be attached to an IV pole. As illustrated in FIG. 1C, a lower portion of the sterile rack 100 may include a tubular receptacle 124 to fit over the top of an IV pole 130 or other support structure. In this manner, the sterile rack 100 may be incorporated into a mobile station, as well as be integrated into a component which serves an additional function in the Essure® procedure. For example, saline bags may also be supported on the IV pole 130 for use in distention of the uterine cavity during the Essure® procedure. In an embodiment, the sterile rack 100 is rotatable 360 degrees on the IV pole 130 in order to provide access to the instruments. For example, the tubular receptacle 124 may rotate around the IV pole 130.

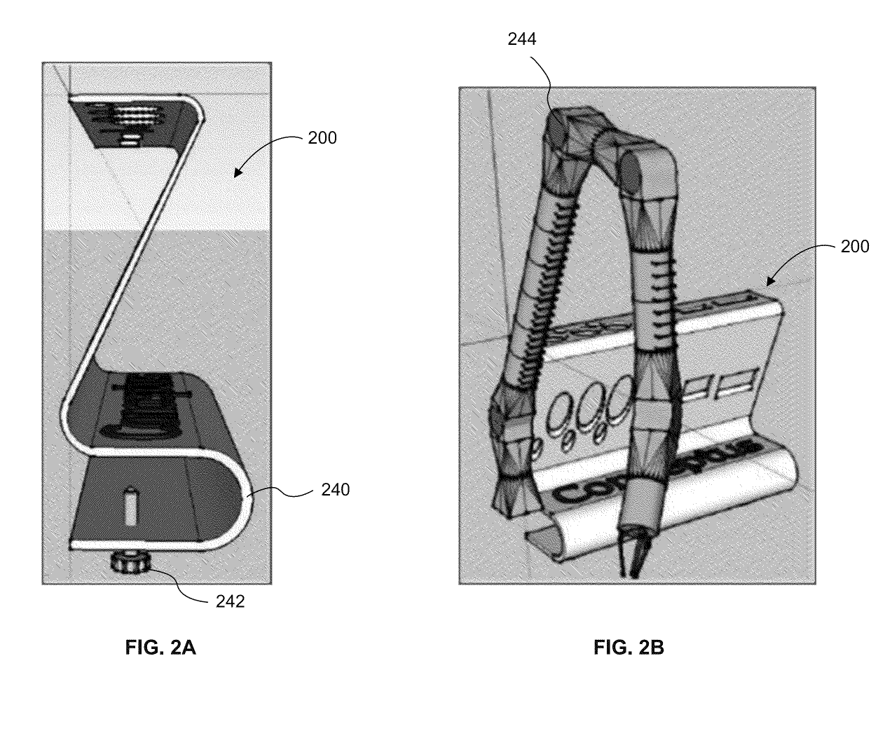

In another embodiment, a sterile rack may be configured to clamp or clip onto a table. For example, referring to FIG. 2A, the lower portion 240 of the sterile rack 200 may include a clamp 242 to clamp onto a table top commonly found in the room in which the procedure is performed. The lower portion of the sterile rack may also be sized to clip onto a table top. Referring the FIG. 2B, the sterile rack 200 may include an adjustable arm clamp 244 which can be configured to clamp onto a variety of surfaces. The arm clamp 244 may be rigid or adjustable to vertically, horizontally, and rotationally position the sterile rack.

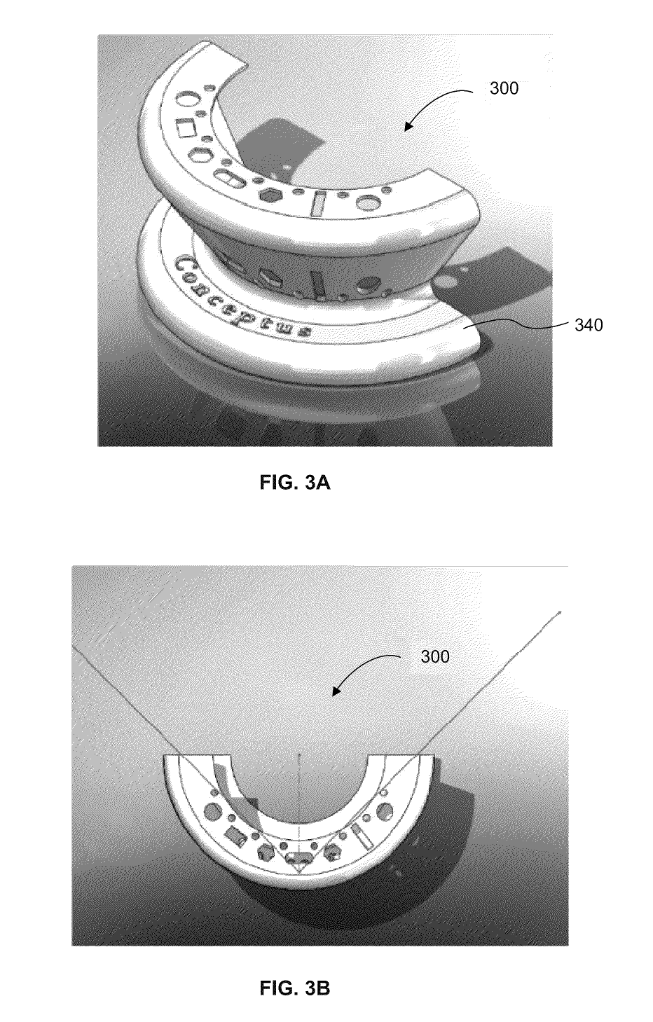

FIG. 3A illustrates a perspective view of a sterile corner rack 300 in accordance with an embodiment of the present invention. As illustrated, the lower portion 340 of the sterile corner rack 300 may be sized to clip onto a table commonly found in the room in which the procedure is performed. The lower portion 340 of the sterile corner rack may also include a clamp to clamp onto a table top corner similar to clamp 242 described with regard to FIG. 2A. Referring to FIG. 3B, the sterile corner rack 300 may be semi-circular to assist with clipping, clamping or otherwise attaching to a table corner which can be a 90 degree corner or rounded corner.

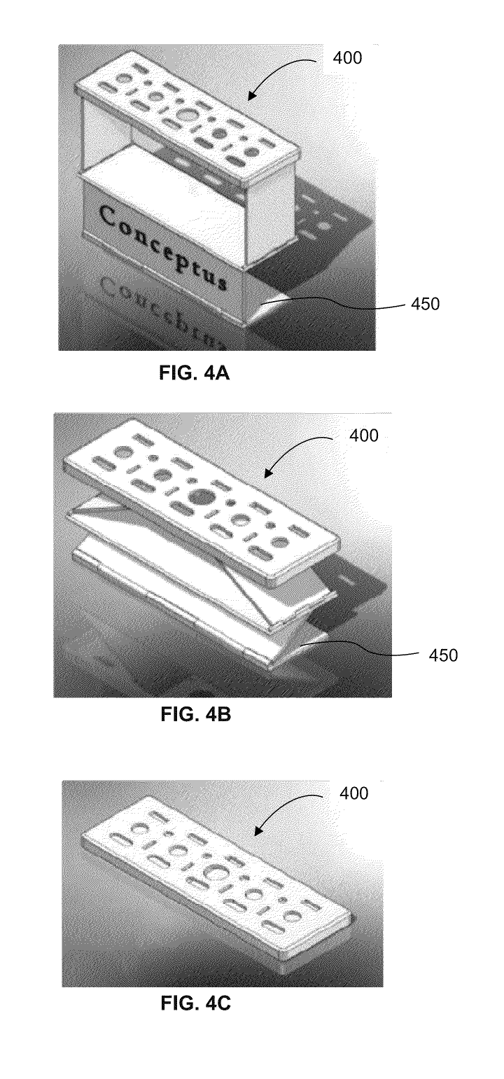

Referring to FIGS. 4A-4C, a sterile rack may also be made to be collapsible for easier storage. As illustrated, the lower surface 450 of the sterile rack 400 is flat and may rest on a table top surface, though any of the other configurations previously described for clipping, clamping or otherwise attaching the sterile rack to an object are compatible with the collapsible sterile rack. Referring now to FIG. 4B, the collapsible sterile rack 400 is illustrated in a partially collapsed configuration, and the collapsible sterile rack 400 is illustrated in a fully collapsed configuration in FIG. 4C.

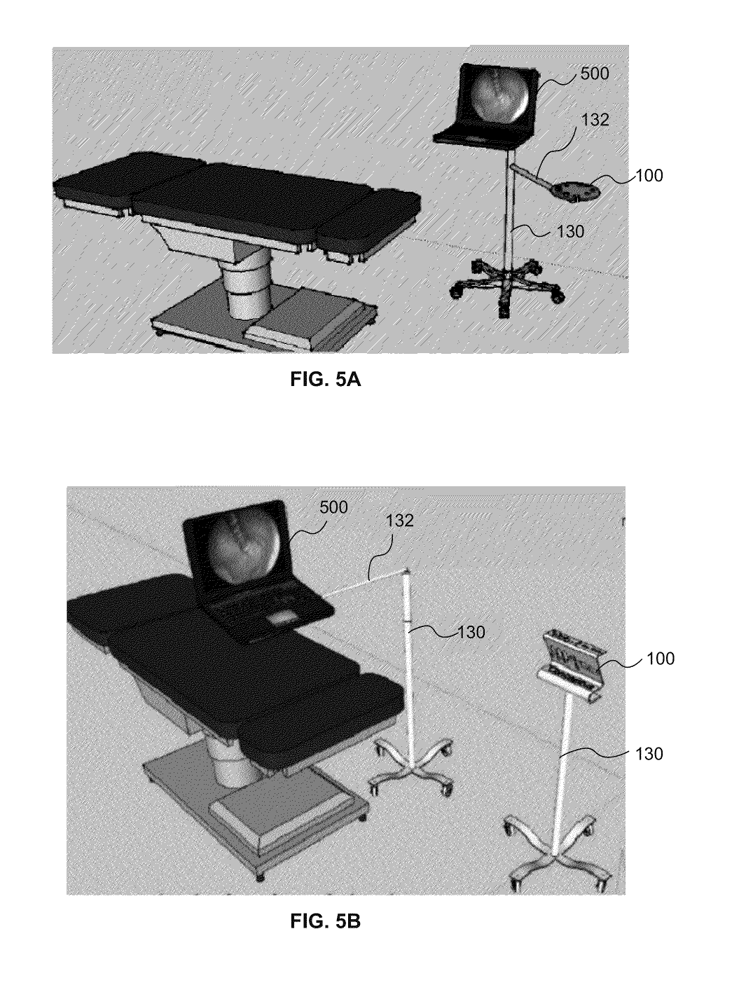

In another embodiment of the present invention, the sterile rack may be included within an office system geared toward the Essure® procedure or anther procedure such as a c-section, OBGYN appointment, child birthing, and other non OBGYN procedure. As illustrated in FIGS. 5A-5B, the office system may include a sterile rack 100, a laptop 500, a camera with mount (not illustrated), a light source (not illustrated) and a mobile station such as an IV pole 130 on wheels. The sterile rack 100 and/or laptop 500 can be attached to mobile station for convenience. For example, as illustrated, the sterile rack 100 or laptop 500 can be attached to the top of the IV pole 130 or to an adjustable arm 132. In an embodiment, the sterile rack 100 is rotatable 360 degrees on the IV pole 130 or adjustable arm 132 in order to provide access to the instruments. The laptop may be useful for providing video recording capabilities and screen capture for pictures. This can allow for a clinical liason or preceptor at a remote location such as a call center or home office to view and assist a doctor performing the procedure. If the doctor has a problem during the procedure, the doctor can contact the clinical liason or preceptor who can see the laptop screen remotely and guide the doctor through the procedure. As illustrated in FIG. 5A, the laptop 500 and sterile rack 100 may be attached to a single mobile station, or IV pole 130. As illustrated in FIG. 5B, the laptop 500 and sterile rack 100 may be attached to separate mobile stations, or IV poles 130.

The sterile racks 100 in accordance with embodiments of the invention may be interchangeable within the office system based upon the specific procedure to be performed with the office system. Where the procedure is the Essure® procedure, the sterile rack may include any of the instruments previously described above. In accordance with embodiments of the invention, the instruments are organized and readily available for the doctor during the procedure. Accordingly, a sterile nurse may not be needed during the procedure. The instruments can be held vertically instead of horizontally, and the doctor can place the instruments easily when needed and not balance parts on the patient. The sterile rack and office system can be used with a variety of procedures, including global endometrial ablation (GEA) procedures commonly performed to treat heavy menstrual bleeding. Interchangeable sterile racks for a variety of procedures can be provided and used with the office system.

In the foregoing specification, various embodiments of the invention have been described. It will, however, be evident that various modifications and changes may be made thereto without departing from the broader spirit and scope of the invention as set forth in the appended claims. The specification and drawings are, accordingly, to be regarded in an illustrative sense rather than a restrictive sense. Hence, the scope of the present invention is limited solely by the following claims.

What is claimed is:

1. A sterile rack comprising:
a plurality of openings, each opening designed to retain a specific instrument in a substantially vertical position for use in a medical procedure.

2. The sterile rack of claim 1, wherein the medical procedure is a transcervical sterilization procedure.

3. The sterile rack of claim 2, wherein the plurality of openings comprises:
a speculum opening
a tenaculum opening;
an introducer opening; and
a delivery system opening.

4. The sterile rack of claim 2, wherein the plurality of openings further comprises:
a graspers opening;
a hysteroscope opening; and
a dilator opening.

5. The sterile rack of claim 4, wherein the plurality of openings comprises:
three dilator openings.

6. The sterile rack of claim 3, wherein the plurality of openings further comprises:
two introducer openings; and
two delivery system openings.

7. The sterile rack of claim 1, further comprising a clamp to stabilize the sterile rack.

8. The sterile rack of claim 7, wherein the sterile rack is configured to be clamped to a table top.

9. The sterile rack of claim 7, comprising an adjustable arm clamp.

10. The sterile rack of claim 1, further comprising a clip to stabilize the sterile rack.

11. The sterile rack of claim 10, wherein the sterile rack is configured to be clipped onto a rounded table corner.

12. The sterile rack of claim 1, wherein the sterile rack includes a tubular receptacle at a lower portion of the sterile rack.

13. The sterile rack of claim 12, wherein the tubular receptacle fits over a top of an IV pole and is rotatable 360 degrees on the IV pole.

14. The sterile rack of claim 1, wherein the sterile rack is collapsible.

15. An office system comprising:
a sterile rack including a plurality of openings, each opening designed to retain a specific instrument in a substantially vertical position for use in a medical procedure;
a laptop;
a camera;
a light source; and
a mobile station.

16. The office system of claim 15, wherein the mobile station comprises an IV pole.

17. The sterile rack of claim 16, wherein the sterile rack includes a tubular receptacle that fits over a top of an IV pole and is rotatable 360 degrees on the IV pole

18. The sterile rack of claim 17, wherein the plurality of openings comprises:
a speculum opening
a tenaculum opening;
an introducer opening; and
a delivery system opening.

19. The sterile rack of claim 18, wherein the plurality of openings further comprises:
a graspers opening;
a hysteroscope opening; and
a dilator opening.

20. The sterile rack of claim 15, wherein the plurality of openings comprises:
a speculum opening
a tenaculum opening;
an introducer opening;
a delivery system opening;
a graspers opening;
a hysteroscope opening; and
a dilator opening.
